(19) United States
(12) Patent Application Publication
(10) Pub. No.: US 2026/0186412 A1
(43) Pub. Date: Jul. 2, 2026

(54) PHOTOACID GENERATOR, PREPARATION METHOD THEREFOR, AND PHOTORESIST COMPOSITION COMPRISING PHOTOACID GENERATOR

(71) Applicant: TienMicro (Shandong) Co., Ltd., Binzhou (CN)

(72) Inventors: Jian XU, Binzhou (CN); Gang CUI, Binzhou (CN); Kunming WANG, Binzhou (CN); Peng GU, Binzhou (CN); Yingying ZHOU, Binzhou (CN); Qiang ZHANG, Binzhou (CN); Guanming ZHUANG, Binzhou (CN)

(21) Appl. No.: 19/436,184

(22) Filed: Dec. 30, 2025

(30) Foreign Application Priority Data
Dec. 31, 2024 (CN) 202411993824.3

(51) Int. Cl. G03F 7/004; C07C 381/12; C07D 213/79; C07D 213/80; C07D 213/803; C08F 220/18; C08F 220/28; G03F 7/00; G03F 7/20

(52) U.S. Cl. CPC G03F 7/0045; C07C 381/12; C07D 213/79; C07D 213/80; C07D 213/803; C08F 220/1808; C08F 220/1809; C08F 220/283; G03F 7/2002; G03F 7/70008; C08F 2800/10

(57) ABSTRACT

The present disclosure relates to a photoacid generator, a preparation method therefor, and a photoresist composition comprising the photoacid generator. The photoacid generator has a structure represented by formula (I). The photoacid generator can be used together with a strongly acidic sulfonium salt photoacid generator to improve resolution and contrast of a lithographic pattern.

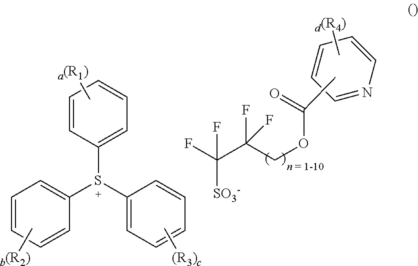

### CROSS-REFERENCE TO RELATED APPLICATIONS

This application claims priority to Chinese application No. 202411993824.3 filed on Dec. 31, 2024, entitled “Photoacid Generator, Preparation Method Therefor, And Photoresist Composition Comprising Photoacid Generator”, the content of which is incorporated herein by reference in its entirety.

### TECHNICAL FIELD

The present application pertains to the technical field of photoresists, and particularly relates to a photoacid generator, a preparation method therefor, and a photoresist composition comprising the photoacid generator.

### BACKGROUND

Photoacid generator (PAG) is an important component of a chemically amplified resist. The photoacid generator generates an acid by photochemical activation. Then the acid diffuses in the photoresist system, and an acid-catalyzed reaction is performed. This process results in decomposition of a protective group on the photoresist resin, eventually generating a carboxylic acid. Subsequently, the carboxylic acid is neutralized by an alkaline developing solution to form an organic salt, which is then dissolved in an aqueous solution, thereby forming a pattern. In the whole process, the photoacid generator maintains its original function while improving the quality and readability of the photoresist.

In the prior art, due to the small volume of anions, the photoacid generator diffuses at a relatively high speed in the photoresist system, which may lead to the following problems: firstly, anions that diffuse at a high speed may cause the generation of line edge roughness (LER) and irregular morphology, and meanwhile, also may result in the phenomenons of photoresist residues, photoresist footing, and the like; secondly, anions that diffuse at a relatively high speed may quickly penetrate into unexposed regions of the photoresist, causing partial decomposition of the photoresist in the unexposed regions, thereby affecting shape accuracy of a lithographic pattern. The anions that diffuse at a high speed can cause an excessively fast reaction rate of the photoresist, so that the photoresist is decomposed unevenly, thereby affecting formation of the lithographic pattern. The anions that diffuse at a high speed are susceptible to environmental factors such as temperature and humidity, which may cause changes in a diffusion rate of a photosensitizer in the photoresist, thereby affecting formation of the lithographic pattern. In summary, the photoacid generator in the prior art has many defects and is difficult to meet the current application requirements in the field of photoresists.

### SUMMARY

The present disclosure provides a photoacid generator that can be applied to lithography (e.g., ArF or KrF lithography) to improve resolution and contrast of a lithographic pattern.

The present disclosure provides the following technical solutions: Provided is a compound represented by formula (I):

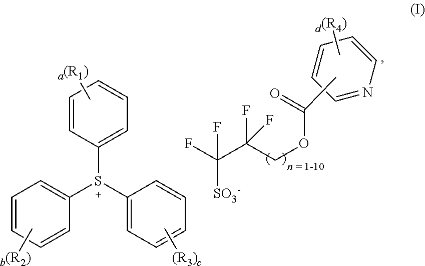

wherein R₁, R₂, R₃, and R₄ are identical or different and are each independently selected from the group consisting of C₁₋₂ alkyl, C₁₋₁₂ alkoxy, C₃₋₂₀ cycloalkyl, and 3- to 20-membered heterocyclyl;
a, b, and c are identical or different and are each independently selected from the group consisting of 0, 1, 2, 3, 4, and 5;
d is selected from the group consisting of 0, 1, 2, 3, and 4;
n is 1, 2, 3, 4, 5, 6, 7, 8, 9, or 10; and
a COO-containing moiety on the left side of pyridine ring in the formula is located at the ortho-, para-, or meta-position of the pyridine ring.

According to an embodiment of the present disclosure, R₁, R₂, R₃, and R₄ are identical or different and are each independently selected from the group consisting of C₁₋₆ alkyl, C₁₋₆ alkoxy, C₃₋₁₂ cycloalkyl, and 3- to 12-membered heterocyclyl;
a, b, and c are identical or different and are each independently selected from the group consisting of 0, 1, and 2; and
d is selected from the group consisting of 0, 1, and 2.

According to an embodiment of the present disclosure, n is 1, 2, 3, or 4.

According to an embodiment of the present disclosure, the compound represented by formula (I) is selected from the group consisting of

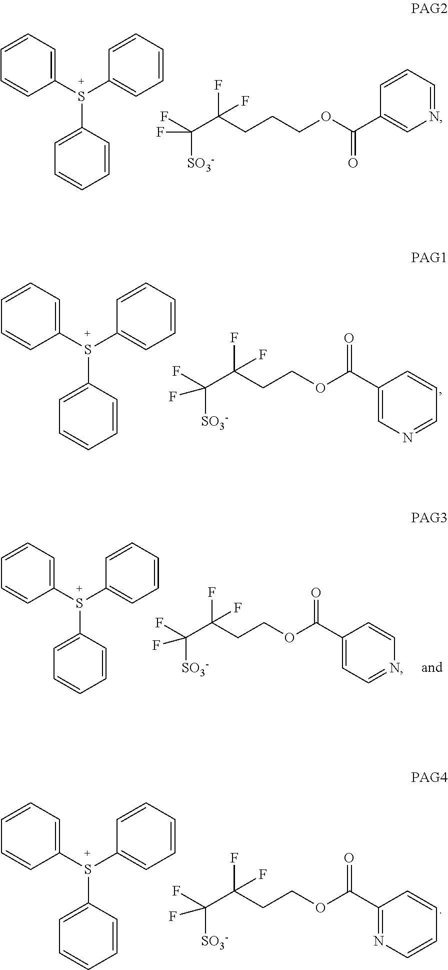

When the compound represented by formula (I) of the present disclosure is used as the photoacid generator for lithography, the introduction of pyridine heterocycle can increase volume of anions of the photoacid generator. The lone pair of electrons on the pyridine ring can better adjust acidity of the photoresist and better control kinetics of the photoresist decomposition process.

The present disclosure further provides a method for preparing the compound represented by formula (I), comprising
method 1: reacting a compound a with a compound b to give the compound represented by formula (I),

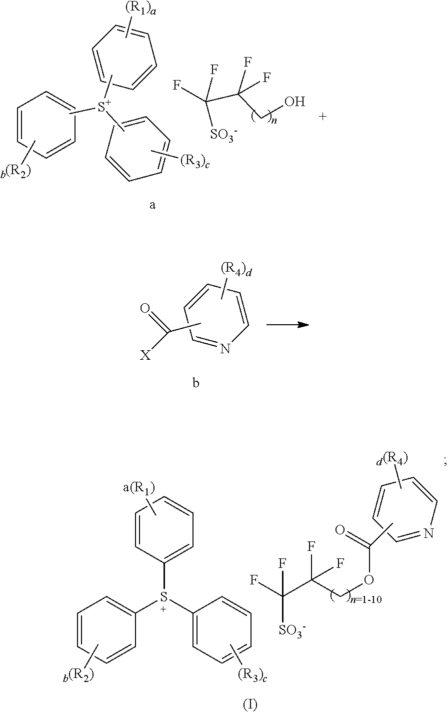

or, method 2: reacting a compound c with a compound d to give the compound represented by formula (I),

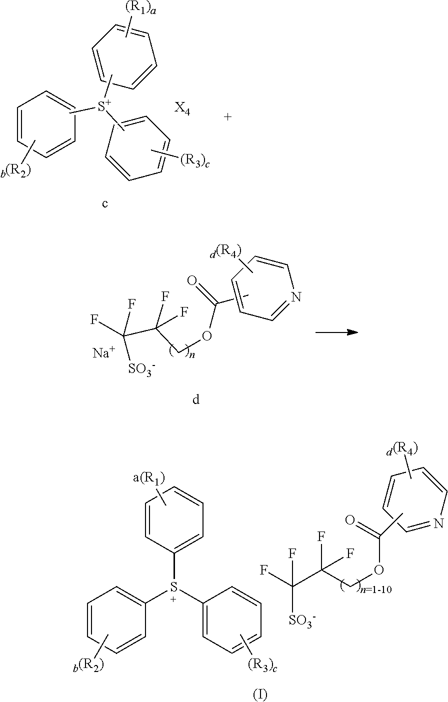

wherein R₁, R₂, R₃, R₄, a, b, c, d, and n are as defined above;
X is halogen, e.g., chlorine; and
X₄ is a halogen ion, e.g., a chloride ion.

The present disclosure further provides use of the compound represented by formula (I) described above as a photoacid generator.

The present disclosure further provides a photoresist composition comprising a polymer resin, and a photoacid generator comprising the compound represented by formula (I) described above. According to an embodiment of the present disclosure, the photoacid generator further comprises a strongly acidic sulfonium salt photoacid generator. Preferably, the strongly acidic sulfonium salt photoacid generator comprises a sulfate ion and a fluorine atom. Preferably, the sulfate ion and the fluorine atom are attached to the same carbon atom. For example, the strongly acidic sulfonium salt photoacid generator is at least one of

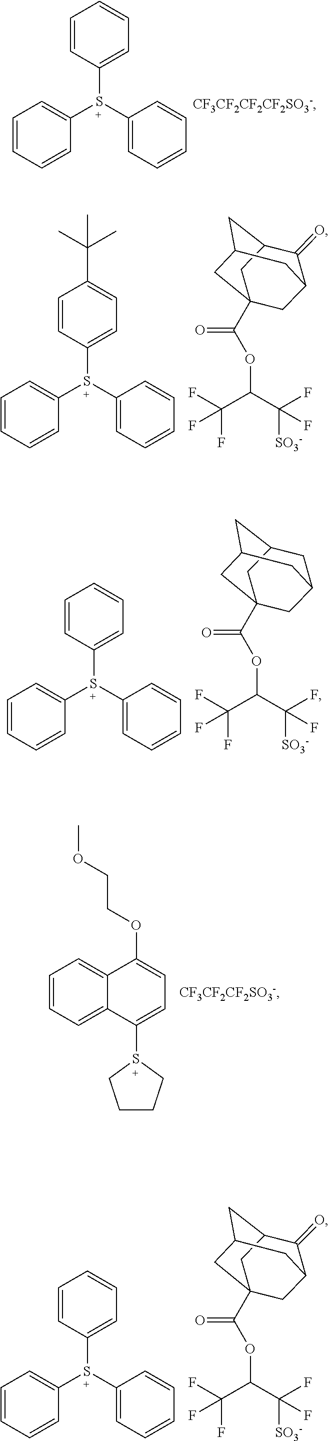

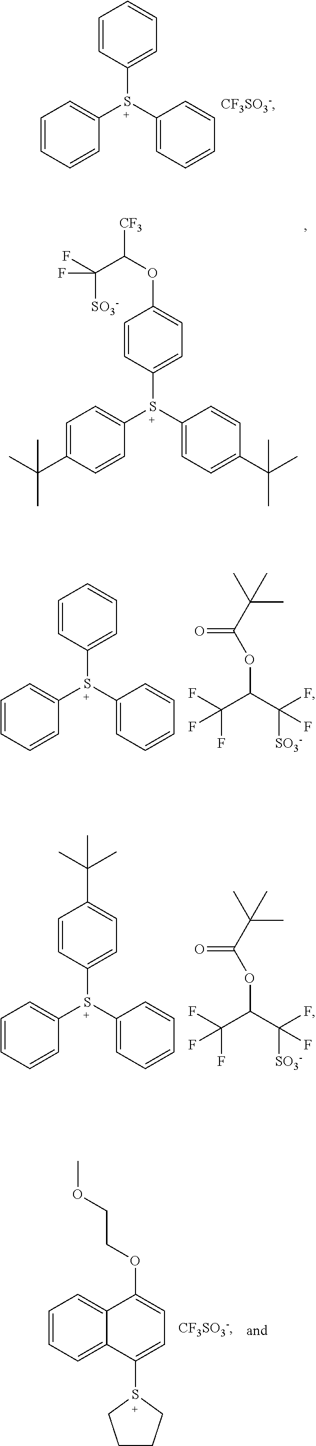

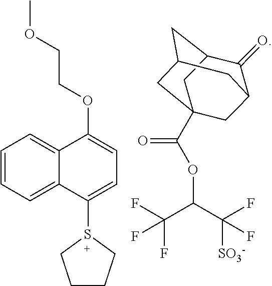

According to an embodiment of the present disclosure, the compound represented by formula I and the strongly acidic sulfonium salt photoacid generator in the photoresist composition are in a mass ratio of 1:1-20, e.g., 1:1-10 or 1:1-5.

In some embodiments of the present disclosure, the polymer resin is a polymer resin applicable for ArF or KrF lithography, e.g., a polymethacrylate resin system, a phenolic resin system, or a resin system of polyhydroxystyrene or a derivative thereof.

According to an embodiment of the present disclosure, the polymer resin is prepared, e.g., by polymerizing at least one of the following monomers:

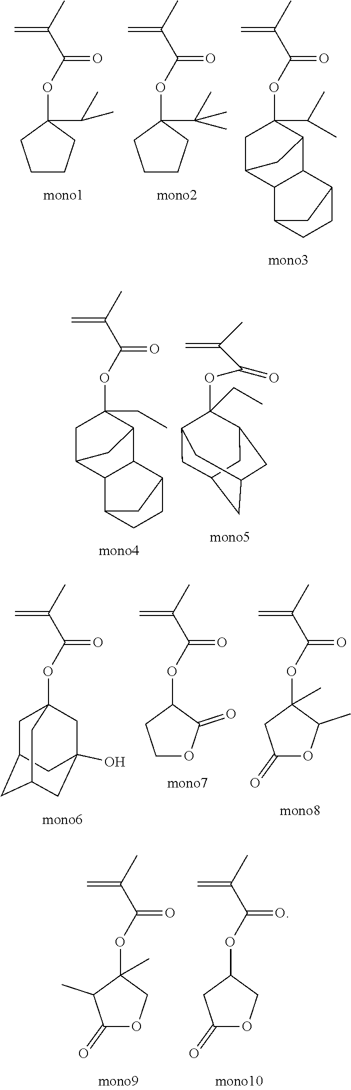

In some embodiments of the present disclosure, the polymer resin is prepared by polymerizing a monomer mono1 or mono2 with a monomer mono7.

According to an embodiment of the present disclosure, the photoresist composition further comprises a solvent. The solvent is selected from the group consisting of propylene glycol methyl ether, ethyl lactate, butyl acetate, propylene glycol methyl ether acetate, propylene glycol dimethyl ether, ethylene glycol monomethyl ether, cyclohexanone, methyl n-amyl ketone, methyl isoamyl ketone, cyclopentanone, ethanol, acetonitrile, isopropanol, acetone, y-butyrolactone, and any combination thereof.

According to an embodiment of the present disclosure, the photoresist composition further comprises a photoacid diffusion inhibitor. The photoacid diffusion inhibitor is a photoacid diffusion inhibitor suitable for a triphenylsulfonium sulfonate system, e.g., triethanolamine.

According to an embodiment of the present disclosure, the photoresist composition is a chemically amplified resist.

According to an embodiment of the present disclosure, the photoresist composition comprises: a polymer resin, a photoacid generator, a photoacid diffusion inhibitor, and a solvent.

According to an embodiment of the present disclosure, the photoresist composition comprises, in parts by mass, 50-200 parts of the polymer resin, 1-30 parts of the photoacid generator, 1-20 parts of the photoacid diffusion inhibitor, and 1000-4000 parts of the solvent.

According to an embodiment of the present disclosure, the photoresist composition comprises, in parts by mass, 80-120 parts of the polymer resin, 10-15 parts of the photoacid generator, 5-12 parts of the photoacid diffusion inhibitor, and 2000-3500 parts of the solvent.

The present disclosure further provides a photoresist coating comprising the photoresist composition described above.

The present disclosure further provides a preparation method for the photoresist coating, which comprises: coating (e.g., spin coating) a substrate with the photoresist composition to give the photoresist coating.

In one embodiment, the substrate is a silicon wafer or the like.

The present disclosure further provides use of the photoresist coating described above in lithography.

In one embodiment, the photoresist coating is used for 193 nm lithography or 248 nm lithography.

### Beneficial Effects

The photoacid generator according to the present disclosure can be used together with a strongly acidic sulfonium salt photoacid generator to improve resolution and contrast of a lithographic pattern. This is mainly because the anion of the photoacid generator according to the present disclosure contains a fluoroalkanesulfonic acid group and a pyridine group; upon exposure, sulfonic acid is generated in the exposed region, which neutralizes pyridine to form a strong acid-weak base salt, imparting weak acidity; meanwhile, the pyridine in the unexposed region is weakly alkaline and can neutralize the sulfonic acid groups diffused from the exposed region, thereby improving exposure contrast.

### Terms and Definitions

The term “C₁₋₁₂ alkyl” should be understood to refer to a linear or branched saturated monovalent hydrocarbyl group having 1-12 carbon atoms, preferably “C₁₋₆ alkyl”. For example, “C₁₋₆ alkyl” refers to linear or branched alkyl group having 1, 2, 3, 4, 5, or 6 carbon atoms. The alkyl is, e.g., methyl, ethyl, propyl, butyl, pentyl, hexyl, isopropyl, isobutyl, sec-butyl, tert-butyl, isopentyl, 2-methylbutyl, 1-methylbutyl, 1-ethylpropyl, 1,2-dimethylpropyl, neopentyl, 1,1-dimethylpropyl, 4-methylpentyl, 3-methylpentyl, 2-methylpentyl, 1-methylpentyl, 2-ethylbutyl, 1-ethylbutyl, 3,3-dimethylbutyl, 2,2-dimethylbutyl, 1,1-dimethylbutyl, 2,3-dimethylbutyl, 1,3-dimethylbutyl, 1,2-dimethylbutyl, or the like, or an isomer thereof.

The term “C₁₋₁₂ alkoxy” refers to —O—C₁₋₁₂ alkyl, where C₁₋₁₂ alkyl is defined as above.

The term “C₃₋₂₀ cycloalkyl” refers to a saturated monocyclic or bicyclic hydrocarbon ring having 3-20 carbon atoms, preferably “C₃₋₁₂ cycloalkyl”. The term “C₃₋₁₂ cycloalkyl” refers to a saturated monovalent monocyclic or bicyclic hydrocarbon ring having 3, 4, 5, 6, 7, 8, 9, 10, 11, or 12 carbon atoms. The C₃₋₁₂ cycloalkyl may be a monocyclic hydrocarbon group, such as cyclopropyl, cyclobutyl, cyclopentyl, cyclohexyl, cycloheptyl, cyclooctyl, cyclononyl or cyclodecyl, or a bicyclic hydrocarbon group, such as a decaline ring.

The term “3- to 20-membered heterocyclyl” refers to a saturated or partially unsaturated monocyclic or bicyclic hydrocarbon ring containing 3 to 20 ring atoms, where one or more of the ring atoms is(are) a heteroatom or atom group selected from the group consisting of N, O, NH, S, S(O), and S(O)₂, excluding a cyclic moiety of —O—O—, —O—S—, or —S—S—, and the remaining ring atom(s) is(are) carbon atom(s). Preferably, the heterocyclyl contains 3 to 12 ring atoms, where 1-4 (e.g., 1, 2, 3, or 4) of the ring atoms are heteroatoms. More preferably, the heterocyclyl contains 3 to 6 (e.g., 3, 4, 5, or 6) ring atoms. The heterocyclyl may be attached to the remainder of the molecule via any one of the carbon atoms or a nitrogen atom (if present) or an oxygen or sulfur atom (particularly where an onium salt is formed). The heterocyclyl may include a fused or bridged ring and/or a spiro ring. Non-limiting examples of a monocyclic heterocyclyl group include azetidinyl, oxetanyl, pyrrolidinyl, imidazolidinyl, tetrahydrofuranyl, tetrahydrothienyl, dihydroimidazolyl, dihydrofuranyl, dihydropyrazolyl, dihydropyrrolyl, dioxolyl, tetrahydropyranyl, pyrrolinyl, piperidyl, piperazinyl, morpholinyl, thiomorpholinyl, dithianyl, trithianyl, homopiperazinyl, diazepanyl, etc.; piperidyl and pyrrolidinyl are preferred. Polycyclic heterocyclyl groups include spiro-ring, fused-ring, and bridged-ring heterocyclyl groups and may also be benzo-fused heterocyclyl groups such as dihydroisoquinolyl. The heterocyclyl may be bicyclic, and its non-limiting examples include hexahydrocyclopenta[c]pyrrol-2(1H)-yl and hexahydropyrrolo[1,2-a]pyrazin-2(1H)-yl. Heterocyclyl may also be partially unsaturated, i.e., it may contain one or more double bonds, and its non-limiting examples include dihydrofuranyl, dihydropyranyl, 2,5-dihydro-1H-pyrrolyl, 4H-[1,3,4]thiadiazinyl, 4,5-dihydrooxazolyl, or 4H-[1,4]thiazinyl.

### BRIEF DESCRIPTION OF THE DRAWINGS

FIG. 1 shows a lithographic pattern of Comparative Example 1.

FIG. 2 shows a lithographic pattern of Example 1.

FIG. 3 shows a lithographic pattern of Example 2.

### DETAILED DESCRIPTION

Unless otherwise stated, the starting materials and reagents used in the following examples are all commercially available products or can be prepared by using known methods.

Preparation of Photoacid Generators

### Preparation Example 1: Preparation Method 1 for PAG1

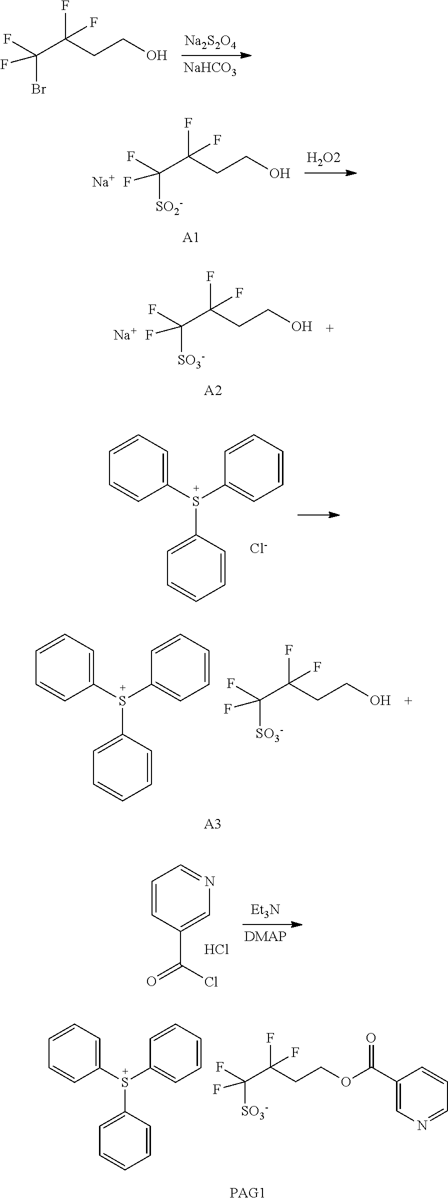

1 g of 4-bromo-3,3,4,4-tetrafluoro-1-butanol, 3 g of sodium dithionite, and 2.5 g of sodium bicarbonate were added to a 250 mL round-bottomed flask, separately, and then 50 g of acetonitrile and 80 g of water were added as reaction solvent. The reaction solution was reacted overnight at 75° C. Heating of the reaction was stopped, and the solvent was removed by rotary evaporation to give crude product A1 as a white solid.

1 g of A1 and 100 g of water were added to a 250 mL round-bottomed flask and purged 3 times with nitrogen to fill the round-bottomed flask with nitrogen, followed by dropwise addition of 5 g of hydrogen peroxide. After the dropwise addition was completed, the mixture was reacted at room temperature for 24 h. After the reaction was terminated, 50 g of dichloromethane was added for extraction, and the reaction solution was washed multiple times with water. Finally, dichloromethane was removed by rotary evaporation to give crude product A2 (0.85 g) as a white solid.

9.88 g of A2 and 11.92 g of triphenylsulfonium chloride were added to a 250 mL round-bottomed flask, and 50 g of dichloromethane and 76 g of deionized water were added. The mixture was purged 3 times with nitrogen to fill the round-bottomed flask with nitrogen, and reacted at room temperature for 24 h. After the reaction was terminated, the reaction solution was washed multiple times with water. Finally, dichloromethane was removed by rotary evaporation to give A3 (16.32 g) as a solid.

4.88 g of A3, 5 g of triethylamine, and 0.2 g of DMAP (4-dimethylaminopyridine) were added to a 250 mL round-bottomed flask, and 130 g of dichloromethane was added. The round-bottomed flask was placed in an ice bath and the mixture was stirred. 2.65 g of nicotinoyl chloride hydrochloride, 0.2 g of triethylamine, and 10 g of dichloromethane were added to a dropping funnel, and the solution in the dropping funnel was added dropwise to the round-bottomed flask. The reaction mixture was stirred overnight. The reaction was quenched with 66 g of water. The solution was washed multiple times with water, and finally was rotary evaporated to give PAG1 (5.5 g) as a white solid. ¹H NMR (400 MHz, CDCl₃) δ=9.17 (d, J=1.7 Hz, 1H), 8.76 (dd, J=4.9, 1.7 Hz, 1H), 8.28 (dt, J=8.0, 1.9 Hz, 1H), 7.79-7.68 (m, 15H), 7.40 (dd, J=7.9, 4.9 Hz, 1H), 4.63 (t, J=6.6 Hz, 2H), 2.90 (tt, J=18.4, 6.6 Hz, 2H).

### Preparation Example 2: Preparation Method 2 for PAG1

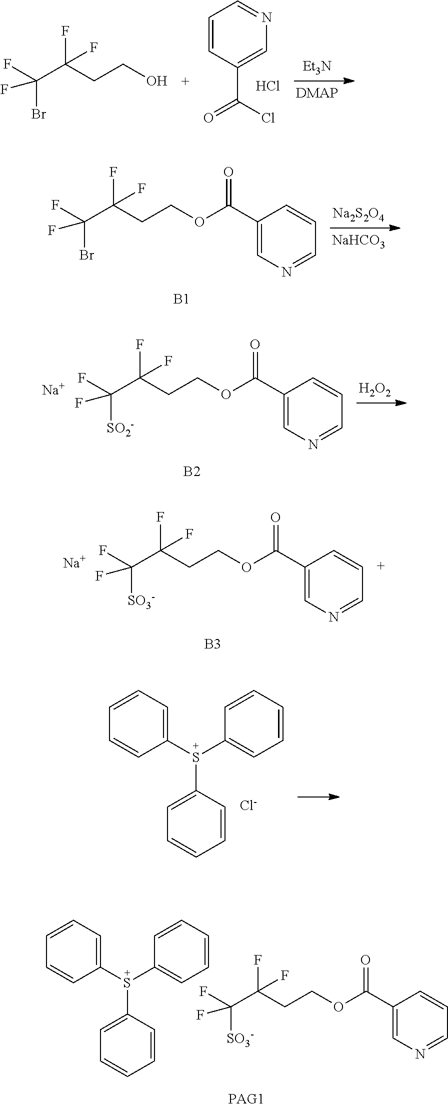

2 g of 4-bromo-3,3,4,4-tetrafluoro-1-butanol, 3 g of triethylamine, and 0.2 g of DMAP (4-dimethylaminopyridine) were added to a 250 mL round-bottomed flask, and 130 g of dichloromethane was added. The round-bottomed flask was placed in an ice bath and the mixture was stirred. 4 g of nicotinoyl chloride hydrochloride, 1 g of triethylamine, and 10 g of dichloromethane were added to a dropping funnel and purged 3 times with nitrogen. The solution of nicotinoyl chloride hydrochloride was added to the round-bottomed flask via the dropping funnel, and the reaction mixture was stirred overnight. The reaction was quenched with 66 g of water. The solution was washed multiple times with water, and finally was rotary evaporated to give crude product B1.

1.65 g of Bi, 3 g of sodium dithionite, and 2 g of sodium bicarbonate were added to a 250 mL round-bottomed flask, separately, and then 50 g of acetonitrile and 80 g of water were added as reaction solvent. The mixture was reacted overnight at 75° C. Heating of the reaction was stopped, and the reaction mixture was naturally cooled to room temperature. The reaction solvent was removed by rotary evaporation. The resultant was dissolved in 50 g of dichloromethane, and then washed 3 times with 50 g of water. Dichloromethane was removed by rotary evaporation to give crude product B2 (1.45 g) as a white solid.

1.45 g of B2 and 100 g of water were added to a 250 mL round-bottomed flask and purged 3 times with nitrogen to fill the round-bottomed flask with nitrogen, followed by dropwise addition of 6 g of hydrogen peroxide. After the dropwise addition was completed, the mixture was reacted at room temperature for 24 h. After the reaction was terminated, the reaction solution was washed multiple times with water. Finally, dichloromethane was removed by rotary evaporation to give B3 (1.2 g) as a white solid.

3.53 g of B3 and 2.98 g of triphenylsulfonium chloride were added to a 250 mL round-bottomed flask, and 50 g of dichloromethane and 76 g of deionized water were added. The mixture was purged 3 times with nitrogen to fill the round-bottomed flask with nitrogen, and reacted at room temperature for 24 h. After the reaction was terminated, the reaction solution was washed multiple times with water. Finally, dichloromethane was removed by rotary evaporation to give PAG1 (5.1 g) as a white solid.

### Preparation Example 3: Preparation Method 1 for PAG2

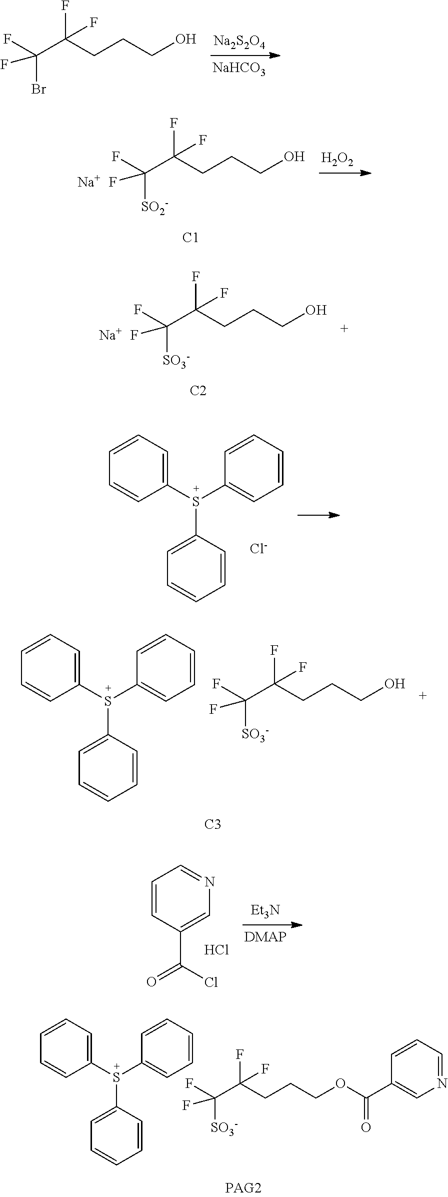

1.2 g of 5-bromo-4,4,5,5-tetrafluoro-1-pentanol, 3 g of sodium dithionite, and 2.5 g of sodium bicarbonate were added to a 250 mL round-bottomed flask, separately, and then 50 g of acetonitrile and 80 g of water were added as reaction solvent. The resulting system was reacted overnight at 75° C. Heating of the reaction was stopped, and the reaction solvent was removed by rotary evaporation to give crude product C1 as a white solid.

1.2 g of C1 and 100 g of water were added to a 250 mL round-bottomed flask and purged 3 times with nitrogen to fill the round-bottomed flask with nitrogen, followed by dropwise addition of hydrogen peroxide. After the dropwise addition was completed, the mixture was reacted at room temperature for 24 h. After the reaction was terminated, 50 g of dichloromethane was added for extraction, and the reaction solution was washed multiple times with water. Finally, dichloromethane was removed by rotary evaporation to give crude product C2.

10.43 g of C2 and 11.92 g of triphenylsulfonium chloride were added to a 250 mL round-bottomed flask, and 50 g of dichloromethane and 76 g of deionized water were added. The mixture was purged 3 times with nitrogen to fill the round-bottomed flask with nitrogen, and reacted at room temperature for 24 h. After the reaction was terminated, the reaction solution was washed multiple times with water. Finally, dichloromethane was removed by rotary evaporation to give C3 (16 g) as a solid.

4.97 g of C3, 5 g of triethylamine, and 0.2 g of DMAP (4-dimethylaminopyridine) were added to a 250 mL round-bottomed flask, and 130 g of dichloromethane was added. The round-bottomed flask was placed in an ice bath and the mixture was stirred. 2.65 g of nicotinoyl chloride hydrochloride, 1 g of triethylamine, and 10 g of dichloromethane were added to a dropping funnel, and the solution of nicotinoyl chloride hydrochloride was added dropwise to the round-bottomed flask via the dropping funnel. The mixture was stirred overnight. The reaction was quenched with 66 g of water. The solution was washed multiple times with water, and finally rotary evaporated to give PAG2 (5.4 g) as a white solid. ¹H NMR (400 MHz, CDCl₃) δ=9.19 (d, J=1.6 Hz, 1H), 8.75 (dd, J=4.9, 1.6 Hz, 1H), 8.30 (dt, J=8.0, 1.9 Hz, 1H), 7.77-7.66 (m, 15H), 7.39 (dd, J=7.9, 4.9 Hz, 1H), 4.37 (t, J=6.6 Hz, 2H), 2.61-2.45 (m, 2H), 2.14-2.04 (m, 2H).

### Preparation Example 4: Preparation Method 2 for PAG2

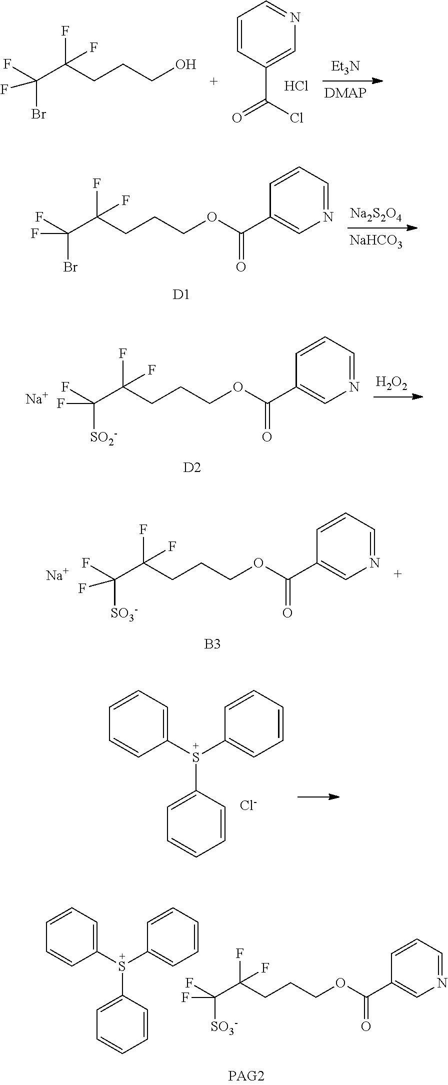

1.3 g of 4-Bromo-3,3,4,4-tetrafluoro-1-pentanol, 3 g of triethylamine, and 0.2 g of DMAP (4-dimethylaminopyridine) were added to a 250 mL round-bottomed flask, and 130 g of dichloromethane was added. The round-bottomed flask was placed in an ice bath and the mixture stirred. 4 g of nicotinoyl chloride hydrochloride, 1 g of triethylamine, and 10 g of dichloromethane were added to a dropping funnel and purged 3 times with nitrogen. The solution of nicotinoyl chloride hydrochloride in dichloromethane was added dropwise to the round-bottomed flask via the dropping funnel, and the mixture was stirred overnight. The reaction was quenched with 66 g of water. The solution was washed multiple times with water, and finally rotary evaporated to give crude product D1 (1.61 g).

1.67 g of D1, 3 g of sodium dithionite, and 2 g of sodium bicarbonate were added to a 250 mL round-bottomed flask, separately, and then 50 g of acetonitrile and 80 g of water were added as reaction solvent. The system was reacted overnight at 75° C. After the reaction was stopped, the reaction solvent was removed by rotary evaporation. The resultant was dissolved in 50 g of dichloromethane, washed 3 times with 50 g of water. Dichloromethane was removed by rotary evaporation to give D2 (1.57 g) as a white solid.

1.51 g of D2 and 100 g of water were added to a 250 mL round-bottomed flask and purged 3 times with nitrogen to fill the round-bottomed flask with nitrogen, followed by dropwise addition of hydrogen peroxide. After the dropwise addition was completed, the mixture was reacted at room temperature for 24 h. After the reaction was terminated, 50 g of dichloromethane was added, and the reaction solution was washed multiple times with water. Finally, dichloromethane was removed by rotary evaporation to give D3 (1.3 g).

3.71 g of D3 and 2.98 g of triphenylsulfonium chloride were added to a 250 mL round-bottomed flask, and 50 g of dichloromethane and 76 g of deionized water were added. The mixture was purged 3 times with nitrogen to fill the round-bottomed flask with nitrogen, and reacted at room temperature for 24 h. The reaction was terminated, and the reaction solution was washed multiple times with water. Finally, dichloromethane was removed by rotary evaporation to give PAG2 (5.0 g).

### Preparation Example 5: Preparation Method 1 for PAG3

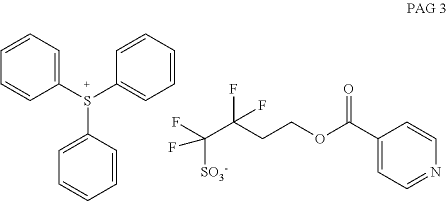

The synthesis methods of steps 1-3 were the same as those in Preparation Example 1. 4.88 g of A3 and 5 g of triethylamine were added to a 250 mL round-bottomed flask, and 130 g of dichloromethane was added. The round-bottomed flask was placed in an ice bath stirred. 2.65 g of isonicotinoyl chloride hydrochloride, 0.2 g of triethylamine, and 10 g of dichloromethane were added to a dropping funnel, and the solution in the dropping funnel was added dropwise to the round-bottomed flask and stirred overnight. The reaction was quenched with 66 g of water. The solution was washed multiple times with water, and finally rotary evaporated to give PAG3 (5.2 g) as a white solid. ¹H NMR (400 MHz, CDCl₃) δ=8.74 (d, J=15.0 Hz, 2H), 7.78 (d, J=15.0 Hz, 2H), 7.79-7.68 (m, 15H), 4.63 (t, J=6.6 Hz, 2H), 2.90 (tt, J=18.4, 6.6 Hz, 2H).

### Preparation Example 6: Preparation Method 2 for PAG 3

2 g of 4-bromo-3,3,4,4-tetrafluoro-1-butanol and 3 g of triethylamine were added to a 250 mL round-bottomed flask, and 130 g of dichloromethane was added. The round-bottomed flask was placed in an ice bath and the mixture was stirred. 4 g of isonicotinoyl chloride hydrochloride, 1 g of triethylamine, and 10 g of dichloromethane were added to a dropping funnel and purged 3 times with nitrogen. The solution of isonicotinoyl chloride hydrochloride was added to the round-bottomed flask via the dropping funnel, and the mixture was stirred overnight. The reaction was quenched with 66 g of water. The solution was washed multiple times with water, and finally rotary evaporated to give the desired crude product.

2.12 g of the crude product, 3 g of sodium dithionite, and 2 g of sodium bicarbonate were added to a 250 mL round-bottomed flask, separately, and then 50 g of acetonitrile and 80 g of water were added as reaction solvent. The mixture was reacted overnight at 75° C. Then heating was stopped, and the reaction mixture was naturally cooled to room temperature. The reaction solvent was removed by rotary evaporation. The resultant was dissolved in 50 g of dichloromethane, and washed 3 times with 50 g of water. Dichloromethane was removed by rotary evaporation to give a crude product (1.62 g) as a white solid.

1.62 g of the crude product obtained in the previous step and 100 g of water were added to a 250 mL round-bottomed flask and purged 3 times with nitrogen to fill the round-bottomed flask with nitrogen, followed by dropwise addition of 6 g of hydrogen peroxide. After the dropwise addition was completed, the mixture was reacted at room temperature for 24 h. After the reaction was terminated, 50 g of dichloromethane was added, and the reaction solution was washed multiple times with water. Finally, dichloromethane was removed by rotary evaporation to give a white solid (1.5 g).

3.53 g of the white solid and 2.98 g of triphenylsulfonium chloride were added to a 250 mL round-bottomed flask, and 50 g of dichloromethane and 76 g of deionized water were added. The mixture was purged 3 times with nitrogen to fill the round-bottomed flask with nitrogen, and reacted at room temperature for 24 h. After the reaction was terminated, the reaction solution was washed multiple times with water. Finally, dichloromethane was removed by rotary evaporation to give PAG3 (4.9 g) as a white solid.

### Preparation Example 7: Preparation Method 1 for PAG4

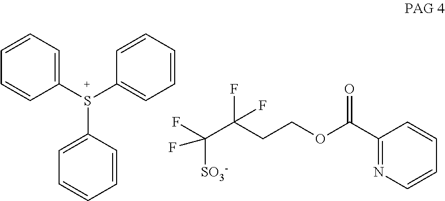

The synthesis methods of steps 1-3 were the same as those in Preparation Example 1. 4.88 g of A3 and 5 g of triethylamine were added to a 250 mL round-bottomed flask, and 130 g of dichloromethane was added. The round-bottomed flask was placed in an ice bath and the mixture was stirred. 2.65 g of pyridine-2-carbonyl chloride hydrochloride, 0.2 g of triethylamine, and 10 g of dichloromethane were added to a dropping funnel, and added dropwise to the round-bottomed flask. The mixture was stirred overnight. The reaction was quenched with 66 g of water. The solution was washed multiple times with water, and finally rotary evaporated to give PAG4 (4.8 g) as a white solid. ¹H NMR (400 MHz, CDCl₃) δ=8.82 (dd, J=14.7, 3.4 Hz, 1H), 8.23 (dd, J=14.7, 3.2 Hz, 1H), 7.89 (dtd, J=47.1, 14.9, 3.2 Hz, 2H), 7.79-7.68 (m, 15H), 4.63 (t, J=6.6 Hz, 2H), 2.90 (tt, J=18.4, 6.6 Hz, 2H).

### Preparation Example 8: Preparation Method 2 for PAG4

2 g of 4-bromo-3,3,4,4-tetrafluoro-1-butanol and 3 g of triethylamine were added to a 250 mL round-bottomed flask, and 130 g of dichloromethane was added. The round-bottomed flask was placed in an ice bath and the mixture was stirred. 4 g of pyridine-2-carbonyl chloride hydrochloride, 1 g of triethylamine, and 10 g of dichloromethane were added to a dropping funnel, and purged 3 times with nitrogen. The solution of pyridine-2-carbonyl chloride hydrochloride was added to the round-bottomed flask via the dropping funnel, and the mixture was stirred overnight. The reaction was quenched with 66 g of water. The solution was washed multiple times with water, and finally rotary evaporated to give the desired crude product.

2.12 g of the crude product, 3 g of sodium dithionite, and 2 g of sodium bicarbonate were added to a 250 mL round-bottomed flask, separately, and then 50 g of acetonitrile and 80 g of water were added as reaction solvent. The mixture was reacted overnight at 75° C. Then heating was stopped, and the reaction mixture was naturally cooled to room temperature. The reaction solvent was removed by rotary evaporation. The resultant was dissolved in 50 g of dichloromethane, and washed 3 times with 50 g of water. Dichloromethane was rotary evaporated to give a crude product (1.7 g) as a white solid.

1.62 g of the crude product obtained in the previous step and 100 g of water were added to a 250 mL round-bottomed flask and purged 3 times with nitrogen to fill the round-bottomed flask with nitrogen, followed by dropwise addition of 6 g of hydrogen peroxide. After the dropwise addition was completed, the mixture was reacted at room temperature for 24 h. After the reaction was terminated, 50 g of dichloromethane was added, and the reaction solution was washed multiple times with water. Finally, dichloromethane was removed by rotary evaporation to give a white solid (1.8 g).

3.53 g of the white solid and 2.98 g of triphenylsulfonium chloride were added to a 250 mL round-bottomed flask, and 50 g of dichloromethane and 76 g of deionized water were added. The mixture was purged 3 times with nitrogen to fill the round-bottomed flask with nitrogen, and reacted at room temperature for 24 h. After the reaction was terminated, the reaction solution was washed multiple times with water. Finally, dichloromethane was removed by rotary evaporation to give PAG4 (4.8 g) as a white solid.

### Synthesis of Resin Type1

98.145 g of 1-(1-methylethyl)cyclopentyl methacrylate and 85.08 g of 2-oxotetrahydrofuran-3-yl methacrylate were added to a 1000 mL round-bottom flask, followed by addition of 200 g of propylene glycol monomethyl ether acetate. The reaction temperature was raised to 80° C., and finally, 5 g of CABN (2-(1-cyano-1-methylethyl)azocarboxamide) and 4 g of n-dodecanethiol were added. The mixture was reacted for 24 h, and then water was added to precipitate the product directly.

The resins Type2 to Type10 were prepared with reference to the preparation method for resin Type1.

The components of active resins Type1 to Type10 are shown in Table 1.

### Preparation of Photoresist Composition

Using the formulation ratio as shown in Table 2 below, 10 g of an active resin, 0.6 g of a photoacid generator, 0.06 g of a photoacid diffusion inhibitor triethanolamine, 54 g of propylene glycol monomethyl ether acetate, and 36 g of cyclohexanone were added to a 150 mL glass flask. The mixture was shaken in the flask at room temperature for 24 h to achieve complete dissolution to prepare a photoresist composition. The photoresist composition was filtered sequentially through a filter having a pore size of 0.22 μm, and the lithography experiment was performed after the completion of filtration.

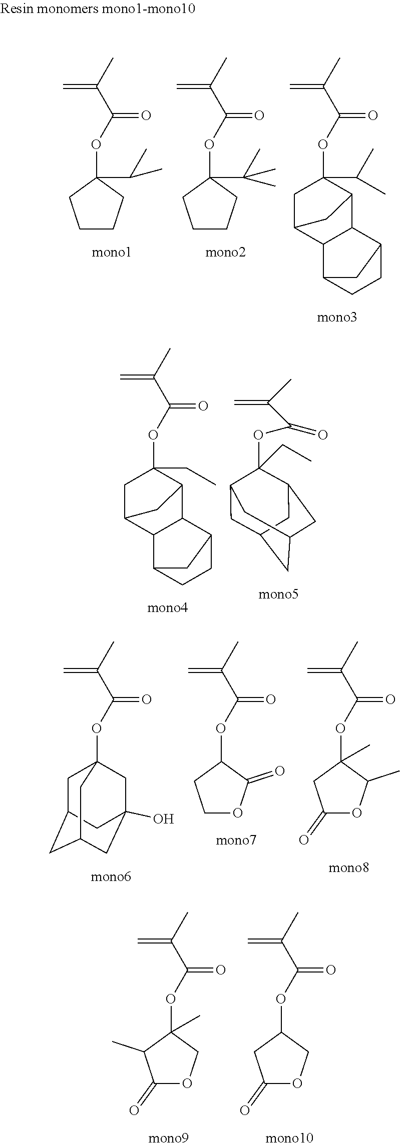

TABLE 1
Components of active resins

| Resin | | | | | | | | | | |
|---|---|---|---|---|---|---|---|---|---|---|
| monomer | | | | | | | | | | |
| (mol) | mono1 | mono2 | mono3 | mono4 | mono5 | mono6 | mono7 | mono8 | mono9 | mono10 |
| Type1 | 0.5 | / | / | / | / | / | 0.5 | / | / | / |
| Type2 | / | 0.5 | / | / | / | / | 0.5 | / | / | / |
| Type3 | 0.2 | / | / | 0.4 | / | 0.1 | / | 0.3 | / | / |
| Type4 | 0.2 | / | / | 0.4 | / | 0.1 | / | / | 0.3 | / |
| Type5 | 0.2 | / | / | 0.4 | / | 0.1 | / | / | / | 0.3 |
| Type6 | 0.2 | / | / | / | 0.4 | 0.1 | / | 0.3 | / | / |
| Type7 | 0.2 | / | / | / | 0.4 | 0.1 | / | / | 0.3 | / |
| Type8 | / | 0.2 | / | 0.4 | / | 0.1 | / | / | / | 0.3 |
| Type9 | / | / | 0.2 | 0.4 | / | / | / | | / | 0.4 |
| Type10 | / | / | 0.2 | 0.4 | / | 0.1 | 0.3 | / | / | / |

“/” in the above table indicates that the corresponding component was not contained.

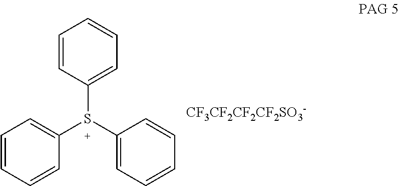

TABLE 2
Addition amount of each component

| | | | | | | | | | Propylene | |
|---|---|---|---|---|---|---|---|---|---|---|
| | | | | | | | | | glycol | |
| | | | | | | | | | monomethyl | |
| Photoresist | PAG | PAG | PAG | PAG | PAG | Resin | Active | Triethanola | ether acetate | Cyclohexanone |
| formulation | 1 (g) | 2 (g) | 3 (g) | 4 (g) | 5 (g) | type | resin (g) | mine (g) | (g) | (g) |
| Comparative | / | / | / | / | 0.6 | Type | 10 | 0.06 | 54 | 36 |
| Example 1 | | | | | | 1 | | | | |
| Example 1 | 0.1 | / | / | / | 0.5 | Type | 10 | 0.06 | 54 | 36 |
| | | | | | | 1 | | | | |
| Example 2 | / | 0.1 | / | / | 0.5 | Type | 10 | 0.06 | 54 | 36 |
| | | | | | | 1 | | | | |
| Example 3 | / | / | 0.1 | / | 0.5 | Type | 10 | 0.06 | 54 | 36 |
| | | | | | | 1 | | | | |
| Example 4 | / | / | / | 0.1 | 0.5 | Type | 10 | 0.06 | 54 | 36 |
| | | | | | | 1 | | | | |
| Example 5 | 0.1 | 0.1 | / | / | 0.4 | Type | 10 | 0.06 | 54 | 36 |
| | | | | | | 1 | | | | |
| Example 6 | 0.1 | / | 0.1 | / | 0.4 | Type | 10 | 0.06 | 54 | 36 |
| | | | | | | 1 | | | | |
| Example 7 | 0.1 | / | / | 0.1 | 0.4 | Type | 10 | 0.06 | 54 | 36 |
| | | | | | | 1 | | | | |
| Example 8 | / | 0.1 | 0.1 | / | 0.4 | Type | 10 | 0.06 | 54 | 36 |
| | | | | | | 1 | | | | |
| Example 9 | 0.1 | / | / | / | 0.5 | Type | 10 | 0.06 | 54 | 36 |
| | | | | | | 2 | | | | |
| Example 10 | / | 0.1 | / | / | 0.5 | Type | 10 | 0.06 | 54 | 36 |
| | | | | | | 2 | | | | |
| Example 11 | / | / | 0.1 | / | 0.5 | Type | 10 | 0.06 | 54 | 36 |
| | | | | | | 2 | | | | |
| Example 12 | / | / | / | 0.1 | 0.5 | Type | 10 | 0.06 | 54 | 36 |
| | | | | | | 2 | | | | |
| Example 13 | 0.1 | 0.1 | / | / | 0.4 | Type | 10 | 0.06 | 54 | 36 |
| | | | | | | 2 | | | | |
| Example 14 | 0.1 | / | 0.1 | / | 0.4 | Type | 10 | 0.06 | 54 | 36 |
| | | | | | | 2 | | | | |
| Example 15 | 0.1 | / | / | 0.1 | 0.4 | Type | 10 | 0.06 | 54 | 36 |
| | | | | | | 2 | | | | |
| Example 16 | / | 0.1 | 0.1 | / | 0.4 | Type | 10 | 0.06 | 54 | 36 |
| | | | | | | 2 | | | | |

“/” in the above table indicates that the corresponding component was not contained.

### ArF Lithography Experiment

Firstly, a 12-inch silicon wafer was spin-coated with an anti-coating layer at a temperature of 205 C for 60 s at a rotation speed of 1500 rpm. Then, the photoresist composition prepared in each of Comparative Example 1 and Examples 1 to 16 was uniformly spin-coated on the silicon wafer at a rotation speed of 1500 rpm with a coating film thickness of 800 A, separately, and the silicon wafer was pre-baked at 90° C. for 1 min and post-baked at 90° C. for 1 min after exposure.

After the silicon wafer was cooled, development was performed using a 2.38% TMAH developing solution for half a minute, and finally, the silicon wafer was rinsed with deionized water for half a minute to form a desired lithographic pattern. The depth of focus (DOF) and exposure latitude (EL) of the resulting pattern are shown in Table 3 below.

TABLE 3
Performance results of the photoresist test

| Photoresist formulation | DOF | EL |
|---|---|---|
| Comparative Example 1 | 0.06 | 7.15% |
| Example 1 | 0.08 | 13.45% |
| Example 2 | 0.1 | 12.47% |
| Example 3 | 0.08 | 12.45% |
| Example 4 | 0.08 | 11.14% |
| Example 5 | 0.08 | 10.42% |
| Example 6 | 0.1 | 11.43% |
| Example 7 | 0.08 | 11.47% |
| Example 8 | 0.08 | 12.45% |
| Example 9 | 0.08 | 12.45% |
| Example 10 | 0.1 | 15.12% |
| Example 11 | 0.08 | 12.07% |
| Example 12 | 0.1 | 11.82% |
| Example 13 | 0.08 | 10.28% |
| Example 14 | 0.08 | 11.42% |
| Example 15 | 0.08 | 10.12% |
| Example 16 | 0.08 | 11.13% |

As can be seen from the above test data for the etching of 5 photoacid generators, compared with the photoresist composition in which only the photoacid generator PAG5 was added, PAG1, PAG2, PAG3, and PAG4 have better resolution and contrast for the same resin. Benefiting from the fact that the anions in PAG1, PAG2, PAG3 and PAG4 contain fluoroalkanesulfonic acid group and pyridinyl group, upon exposure, sulfonic acid generated in the exposed region can neutralize pyridine to form a strong acid-weak base salt, imparting weak acidity; meanwhile, pyridine in the unexposed region has weak basicity and can neutralize sulfonic acid group diffused from the exposed region, thereby improving exposure contrast.

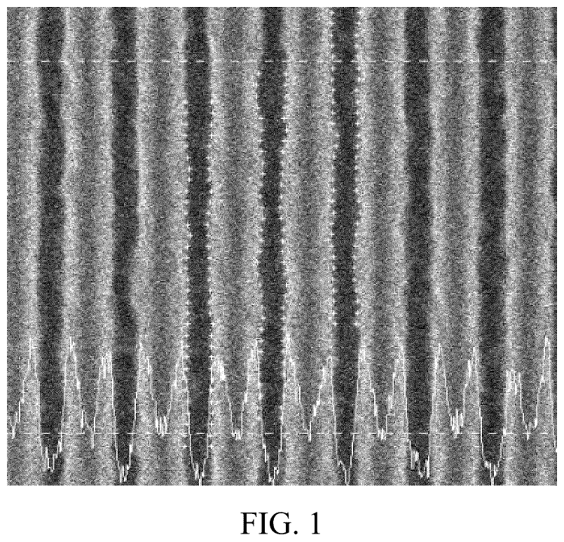

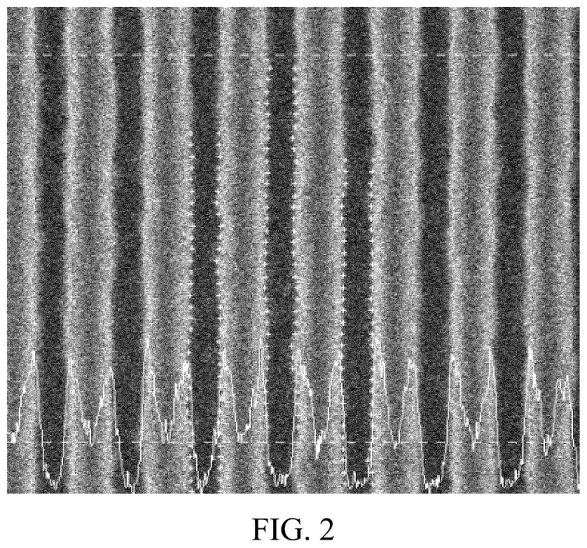

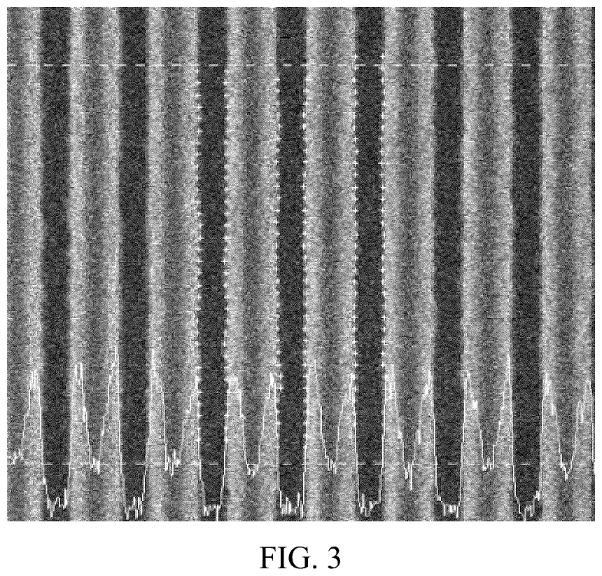

FIG. 1 shows a lithographic pattern of Comparative Example 1, FIG. 2 shows a lithographic pattern of Example 1, and FIG. 3 shows a lithographic pattern of Example 2. As can be seen by comparing FIGS. 1 to 3, with respect to the photoresist formulation in which only PAG5 was added, the photoresist formulation in which PAG1 or PAG2 was additionally added can significantly improve EL (exposure latitude) and DOF (depth of focus).

The photoacid generator of the present application has the same effect in other lithography processes, such as KrF.

The embodiments of the present disclosure have been described above. However, the embodiments described above are not intended to limit the present disclosure. Any modification, equivalent replacement, improvement, and the like made without departing from the spirit and principle of the present disclosure shall fall within the protection scope of the present disclosure.

1. A compound represented by formula (I): 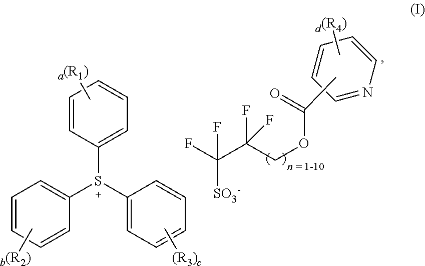
wherein R₁, R₂, R₃, and R₄ are identical or different and are each independently selected from the group consisting of C₁₋₁₂ alkyl, C₁₋₁₂ alkoxy, C₃₋₂₀ cycloalkyl, and 3- to 20-membered heterocyclyl;
a, b, and c are identical or different and are each independently selected from the group consisting of 0, 1, 2, 3, 4, and 5;
d is selected from the group consisting of 0, 1, 2, 3, and 4;
n is 1, 2, 3, 4, 5, 6, 7, 8, 9, or 10; and
a COO-containing moiety on the left side of pyridine ring in the formula is located at the ortho-, para-, or meta-position of the pyridine ring.

2. The compound according to claim 1, wherein R₁, R₂, R₃, and R₄ are identical or different and are each independently selected from the group consisting of C₁₋₆ alkyl, C₁₋₆ alkoxy, C₃₋₁₂ cycloalkyl, and 3- to 12-membered heterocyclyl;
a, b, and c are identical or different and are each independently selected from the group consisting of 0, 1, and 2;
d is selected from the group consisting of 0, 1, and 2; and
n is 1, 2, 3, or 4.

3. The compound according to claim 1, wherein the compound represented by formula (I) is selected from the group consisting of

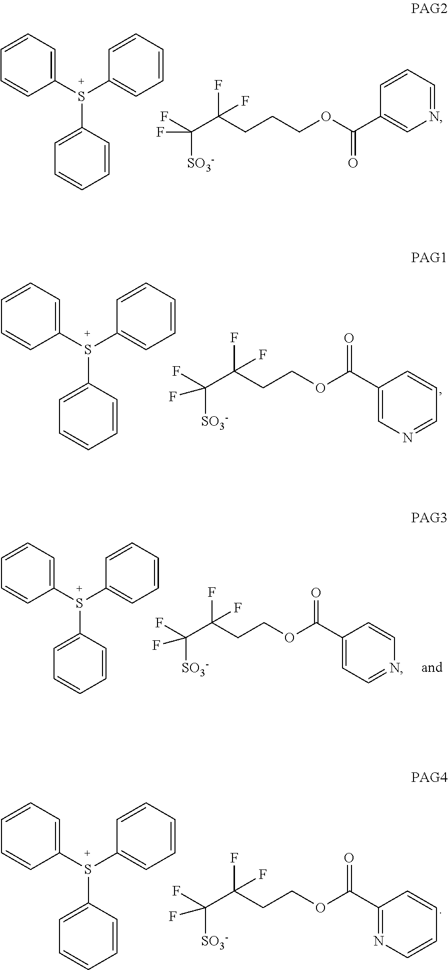

4. A method for preparing the compound according to claim 1, comprising
method 1: reacting a compound a with a compound b to give the compound represented by formula (I), 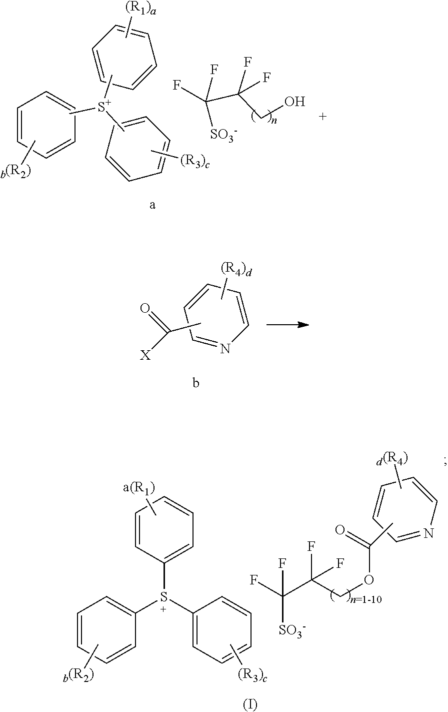
or,
method 2: reacting a compound c with a compound d to give the compound represented by formula (I), 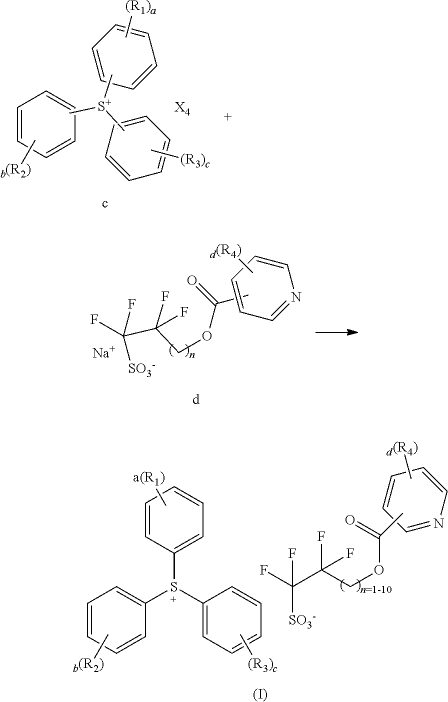
wherein R₁, R₂, R₃, R₄, a, b, c, d, and n are as defined in claim 1;
X is halogen, and X₄ is a halogen ion.

5. Use of the compound according to claim 1 as a photoacid generator.

6. A photoresist composition, comprising a polymer resin and a photoacid generator, wherein the photoacid generator comprises the compound of formula (I) according to claim 1.

7. The photoresist composition according to claim 6, wherein the photoacid generator further comprises a strongly acidic sulfonium salt photoacid generator.

8. The photoresist composition according to claim 7, wherein the strongly acidic sulfonium salt photoacid generator is at least one of

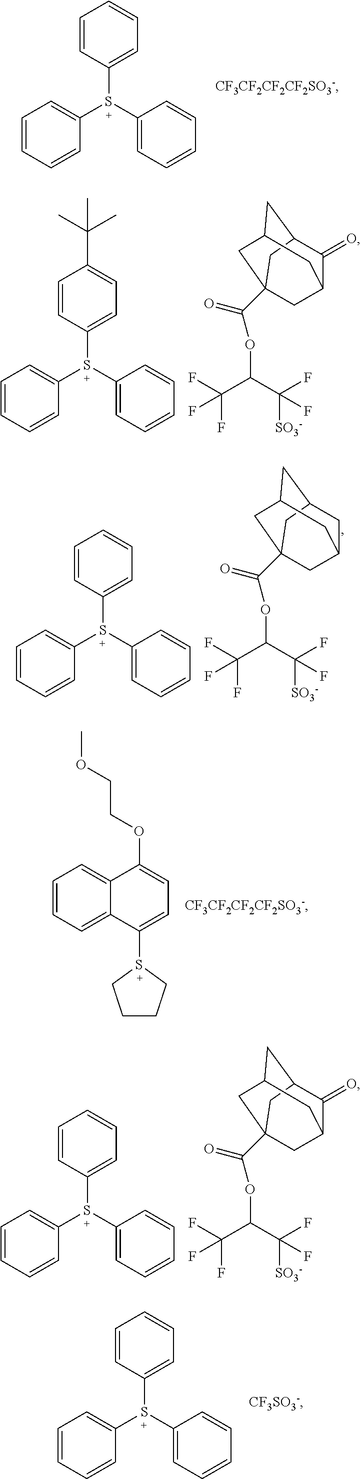

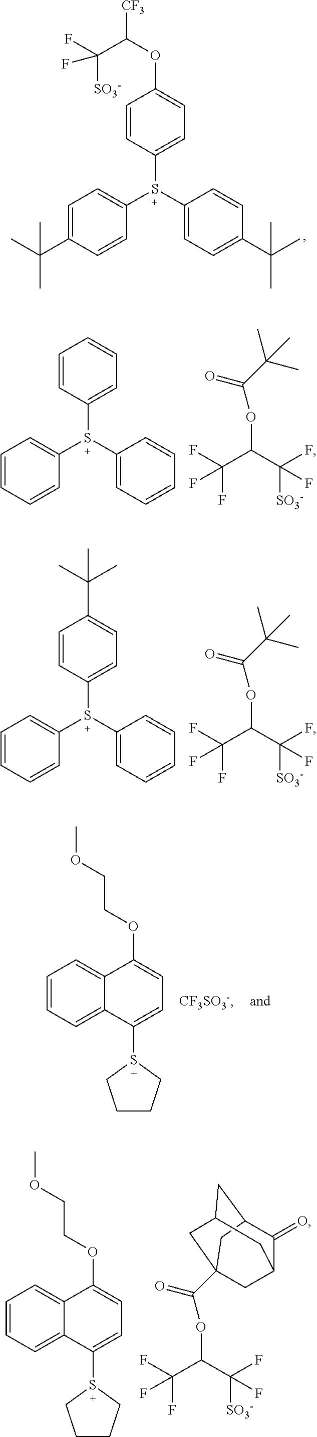

9. The photoresist composition according to claim 7, wherein the compound represented by formula I and the strongly acidic sulfonium salt photoacid generator in the photoresist composition are in a mass ratio of 1:1-20, 1:1-10, or 1:1-5.

10. The photoresist composition according to claim 6, wherein the polymer resin is a polymethacrylate resin system, a phenolic resin system, or a resin system of polyhydroxystyrene or a derivative thereof.

11. The photoresist composition according to claim 6, wherein the polymer resin is prepared by polymerizing at least one of

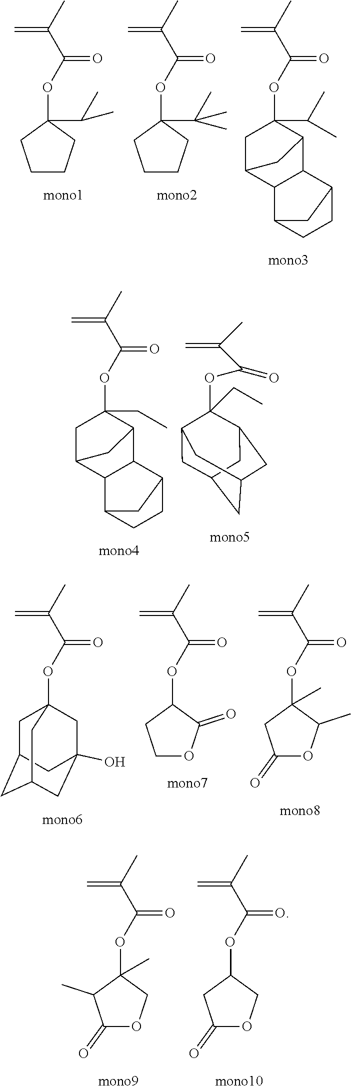

12. The photoresist composition according to claim 6, further comprising a solvent selected from the group consisting of propylene glycol methyl ether, ethyl lactate, butyl acetate, propylene glycol methyl ether acetate, propylene glycol dimethyl ether, ethylene glycol monomethyl ether, cyclohexanone, methyl n-amyl ketone, methyl isoamyl ketone, cyclopentanone, ethanol, acetonitrile, isopropanol, acetone, y-butyrolactone, and any combination thereof.

13. The photoresist composition according to claim 6, further comprising a photoacid diffusion inhibitor.

14. The photoresist composition according to claim 6, comprising, in parts by mass, 50-200 parts of the polymer resin, 1-30 parts of the photoacid generator, 1-20 parts of a photoacid diffusion inhibitor, and 1000-4000 parts of a solvent.

15. A photoresist coating, comprising the photoresist composition according to claim 6.

16. A method for preparing the photoresist coating according to claim 15, comprising coating a substrate with the photoresist composition to give the photoresist coating.

17. Use of the photoresist coating according to claim 15 in lithography.

18. The use according to claim 17, wherein the photoresist coating is used for 193 nm lithography or 248 nm lithography.
